(19) United States
(12) Patent Application Publication
(10) Pub. No.: US 2023/0312465 A1
(43) Pub. Date: Oct. 5, 2023

(54) A PROCESS FOR PREPARATION OF CLETHODIM

(71) Applicant: UPL LIMITED, Mumbai (IN)

(72) Inventors: Rakesh Bhikubhai Patel, Mumbai (IN); Digish Manubhai Panchal, Mumbai (IN)

(21) Appl. No.: 18/041,159

(22) PCT Filed: Aug. 11, 2021
(86) PCT No.: PCT/IB2021/057397

§ 371 Date: Feb. 9, 2023

(30) Foreign Application Priority Data
Aug. 11, 2020 (IN) 202021034499

(51) Int. Cl. C07C 323/47

(52) U.S. Cl. CPC C07C 323/47; C07C 2601/16

(57) ABSTRACT

The present invention provides a process for the preparation of clethodim by reacting 5-[2-(ethylthio)propyl]-2-(1-oxopropyl)-1,3-cyclohexanedione with O-(3-chloro-2-propen-1-yl)hydroxylamine in the absence of a solvent. A continuous flow process for preparing clethodim is also provided.

### FIELD OF INVENTION

The present invention relates to a process for the preparation of clethodim. The present invention more particularly relates to a process for the preparation of clethodim in the absence of a solvent.

### BACKGROUND OF THE INVENTION

Clethodim is a systemic post-emergence herbicide used to control annual and perennial grasses in a wide variety of broad leaf crops. Clethodim is a fatty acid synthesis inhibitor and belongs to substance group of cyclohexanedione oxime.

WO8701699 disclose preparation of clethodim by reacting 5-[2-(ethylthio)propyl]-2-(1-oxopropyl)-1,3-cyclohexanedione with O-(3-chloro-2-propen-1-yl)hydroxylaminein presence of sodium methoxide and methanol.

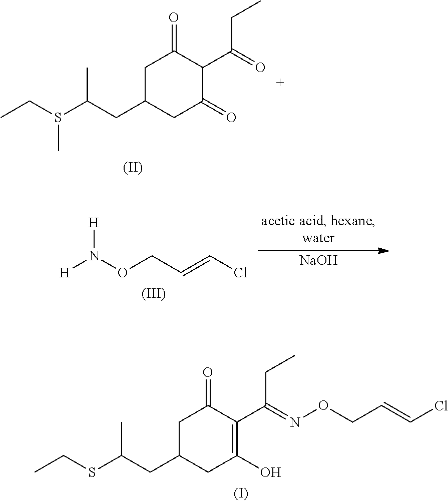

CN107162945 describes preparation of clethodim by reacting 5-[2-(ethylthio)propyl]-2-(1-oxopropyl)-1,3-cyclohexanedione with O-(3-chloro-2-propen-1-yl)hydroxylaminein petroleum ether solvent.

The processes described in the prior art are carried out in solvents which are hazardous to environment.

Therefore, there is a need to develop an improved process for preparation of clethodim.

### OBJECT OF THE INVENTION

It is an object of the present invention to provide a process for the preparation of clethodim which is simple and environmentally friendly.

It is another object of the present invention to provide a process for the preparation of clethodim in high yield and high purity.

It is another object of the present invention to provide a process for the preparation of clethodim which is economical.

An object of the present invention is to overcome or ameliorate at least one of the above disadvantages of the prior art processes and to provide a process for preparation of clethodim by a continuous process.

### SUMMARY OF THE INVENTION

The present invention provides a process for the preparation of clethodim comprising:

reacting 5-[2-(ethylthio)propyl]-2-(1-oxopropyl)-1,3-cyclohexanedione of formula (II)

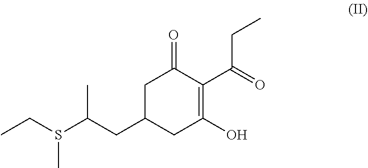

with O-(3-chloro-2-propen-1-yl)hydroxylamine of formula (III)

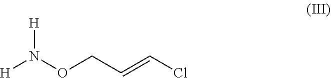

in the absence of a solvent.

The present invention provides a continuous flow process for preparing clethodim, said process comprising:
a) charging 5-[2-(ethylthio)propyl]-2-(1-oxopropyl)-1,3-cyclohexanedione into a microreactor through a first dosing line;
b) charging O-(3-chloro-2-propen-1-yl) hydroxylamine into the microreactor through a second dosing line;
c) reacting 5-[2-(ethylthio)propyl]-2-(1-oxopropyl)-1,3-cyclohexanedione with O-(3-chloro-2-propen-1-yl)hydroxylamine in the absence of a solvent in the microreactor to obtain clethodim.

### BRIEF DESCRIPTION OF FIGURES

The drawings described herein are for illustrative purposes only of selected embodiments and not all possible implementations and are not intended to limit the scope of the present disclosure.

FIG. 1: Plug flow reactor diagram for producing clethodim

FIG. 2: HPLC chromatogram of clethodim

### DETAILED DESCRIPTION OF THE INVENTION

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

For the purposes of the following detailed description, it is to be understood that the invention may assume various alternative variations and step sequences, except where expressly specified to the contrary. Moreover, other than in any operating examples, or where otherwise indicated, all numbers expressing, for example, quantities of materials/ingredients used in the specification are to be understood as being modified in all instances by the term “about”. The term “about” shall be interpreted to mean “approximately” or “reasonably close to” and any statistically insignificant variations there from.

Thus, before describing the present invention in detail, it is to be understood that this invention is not limited to particularly exemplified systems or process parameters that may of course, vary. It is also to be understood that the terminology used herein is for the purpose of describing particular embodiments of the invention only and is not intended to limit the scope of the invention in any manner. The use of examples anywhere in this specification including examples of any terms discussed herein is illustrative only, and in no way limits the scope and meaning of the invention or of any exemplified term. Likewise, the invention is not limited to various embodiments given in this specification.

It must be noted that, as used in this specification, the singular forms “a,” “an” and “the” include plural referents unless the content clearly dictates otherwise. The terms “preferred” and “preferably” refer to embodiments of the invention that may afford certain benefits, under certain circumstances.

As used herein, the terms “comprising” “including,” “having,” “containing,” “involving,” and the like are to be understood to be open-ended, i.e., to mean including but not limited to. Inventors of the present invention have found out that clethodim can be prepared by reacting 5-[2-(ethylthio)propyl]-2-(1-oxopropyl)-1,3-cyclohexanedione with O-(3-chloro-2-propen-1-yl)hydroxylamine (CPHA) in the absence of a solvent.

In another aspect the present invention provides a method and system for the preparation of clethodim particularly in a microreactor system.

Hereunder, the embodiments of the present invention are described in detail.

In an aspect the present invention provides a process for the preparation of clethodim of formula (I), said process comprising:

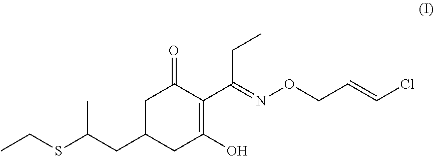 [SMILES: CCSC(C)CC1CC(=O)C(/C(CC)=N/OC/C=C/Cl)=C(O)C1]

reacting 5-[2-(ethylthio)propyl]-2-(1-oxopropyl)-1,3-cyclohexanedione of formula (II)

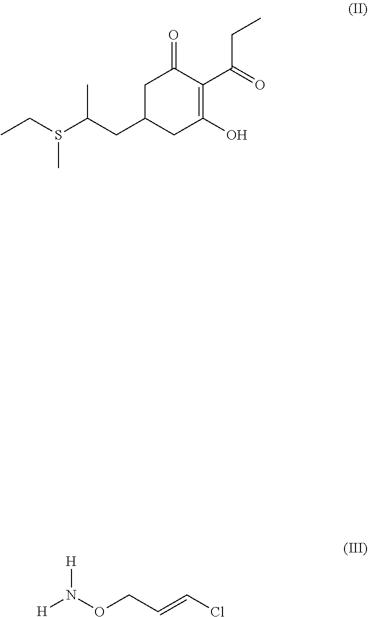

with O-(3-chloro-2-propen-1-yl)hydroxylamine (III) in the absence of a solvent.

In an embodiment, the molar ratio of 5-[2-(ethylthio)propyl]-2-(1-oxopropyl)-1,3-cyclohexanedione to O-(3-chloro-2-propen-1-yl)hydroxylamine is from about 1.0:1.0 to about 1.0:5.0.

In a preferred embodiment, the molar ratio of 5-[2-(ethylthio)propyl]-2-(1-oxopropyl)-1,3-cyclohexanedione to O-(3-chloro-2-propen-1-yl)hydroxylamine is from about 1.0:1.0 to about1.0:1.3.

In a most preferred embodiment, the molar ratio of 5-[2-(ethylthio)propyl]-2-(1-oxopropyl)-1,3-cyclohexanedione to O-(3-chloro-2-propen-1-yl)hydroxylamine is from about 1.0:1.0 to about 1.0:1.05.

In an embodiment of the present invention, the reaction is carried out at a temperature from about 20° C. to about 70° C.

In a specific embodiment of the present invention, the reaction is carried out at a temperature from about 40° to about 70° C.

In specific embodiment the present invention includes a batch process or continuous process for preparation of clethodim.

In an embodiment, the present process for preparing clethodim according to the present invention is a batch process.

In an embodiment, the present process for preparing clethodim according to the present invention is a continuous process.

In an embodiment of the present invention there is provided a continuous flow process for preparing clethodim, said process comprising:
a) charging 5-[2-(ethylthio)propyl]-2-(1-oxopropyl)-1,3-cyclohexanedioneinto a microreactor through a first dosing line;
b) charging O-(3-chloro-2-propen-1-yl) hydroxylamine into the microreactor through a second dosing line;
c) reacting 5-[2-(ethylthio)propyl]-2-(1-oxopropyl)-1,3-cyclohexanedionewith O-(3-chloro-2-propen-1-yl)hydroxylaminein the absence of a solvent in the microreactor.

In an embodiment, the flow rate of 5-[2-(ethylthio)propyl]-2-(1-oxopropyl)-1,3-cyclohexanedione flowing from first dosing line varies from about 1 ml/min to about 20 ml/min in a reactor up to 50 ml capacity.

In a preferred embodiment, the flow rate of 5-[2-(ethylthio)propyl]-2-(1-oxopropyl)-1,3-cyclohexanedione flowing from first dosing line varies from about 2.0 ml/min to about 5.0 ml/min in a reactor up to 50 ml capacity.

In another embodiment, the flow rate of O-(3-chloro-2-propen-1-yl)hydroxylamine flowing from second dosing line varies from 1 ml/min to 20 ml/min in a reactor up to 50 ml capacity.

In a preferred embodiment, the flow rate of O-(3-chloro-2-propen-1-yl)hydroxylamine flowing from first dosing line varies from 2.0 ml/min to 5.0 ml/min in a reactor up to 50 ml capacity.

In an embodiment of the present invention, the reaction is carried out at temperature ranging from about 20° C. to about90° C.

In a preferred embodiment of the present invention, the reaction is carried out at temperature ranging from about 50° C. to about 70° C.

In an embodiment of the present invention, the residence time for the reaction is ranging from about 1 minutes to 20 minutes in a reactor up to 50 ml capacity.

In a preferred embodiment of the present invention, the residence time for the reaction is ranging from about 5 minutes to 10 minutes in a reactor up to 50 ml capacity.

In an embodiment the continuous flow process for preparation of clethodim according to the present invention is carried out using a Plug Flow Reactor.

In an embodiment the continuous flow process for preparation of clethodim according to the present invention is carried out using a reactor selected from Corning G1 flow reactor, Chemtrix MR-260 Flow reactor, or LTF reactor.

According to an embodiment of the present invention, the volume of microreactors for carrying out the continuous flow process for the synthesis of clethodim at laboratory scale are selected from various capacity range of 1 ml, 10 ml, 50ml, 100 ml and the like based on desired output volume of clethodim.

According to an embodiment of the present invention, the volume of microreactors for carrying out the continuous flow process for the synthesis of clethodim at commercial scale are selected from various capacity range of 1L, 10 L, 50 L, 100 L, 500 L, 1000 L, 2000 L, 5000 L, 50000 L and more which can be based on desired output volume of clethodim.

In an embodiment, clethodim produced by a continuous process comprising a system for reacting 5-[2-(ethylthio)propyl]-2-(1-oxopropyl)-1,3-cyclohexanedione with O-(3-chloro-2-propen-1-yl)hydroxylamine (III) in the absence of a solvent.

In a specific embodiment the present invention provides a system comprising a microreactor wherein 5-[2-(ethylthio)propyl]-2-(1-oxopropyl)-1,3-cyclohexanedione is reacted with O-(3-chloro-2-propen-1-yl)hydroxylamine (III) in the absence of a solvent to obtain clethodim.

The process for the preparation of clethodim according to the present invention is illustrated by, but not limited to, the following description and the figures referred therein.

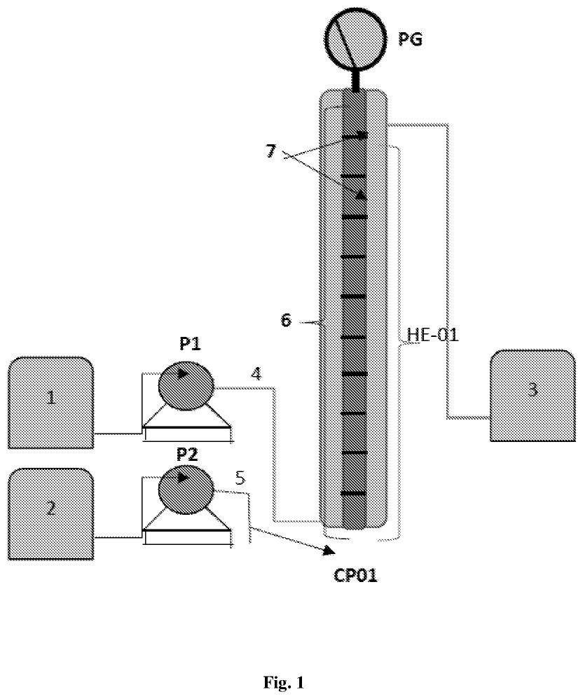

Referring to FIG. 1, the microreactor described is a Plug Flow Reactor (PFR) with reaction vessel (CP-01) for preparation of clethodim. Heating element HE (HE-01) is attached to the reaction vessel (CP-01) to provide requisite temperature indicated by temperature sensor (7). 5-[2-(ethylthio)propyl]-2-(1-oxopropyl)-1,3-cyclohexanedione is introduced in the reactor through first dosing line (4) while O-(3-chloro-2-propen-1-yl)hydroxylamine is introduced through a second dosing line (5). Feed container (1) and (2) are connected to reaction vessel (CP01) through dosing lines (4) and (5) respectively and holds the reactants separately. Pumps P1 and P2 are attached to these dosing lines such that it drives the reactants contained in the feed containers (1) and (2) to the reactor (CP-01). First dosing line (4), is connected to the reactor (CP-01) via pump (P1). Second dosing line (5), is connected to the reactor (CP-01) via pump (P2). The pressure element PG is connected to the reaction vessel (CP-01) to provide pressure indication during reaction. The reaction vessel (CP-01) is connected to collector vessel (3) in which the clethodim is collected.

Although the subject matter has been described in considerable detail with reference to certain preferred embodiments thereof, other embodiments are possible. As such, the spirit and scope of the disclosure should not be limited to the description of the preferred embodiment contained therein.

In an embodiment the present invention provides a system comprising a microreactor unit for producing clethodim by a continuous flow process wherein the process is conducted in the absence of a solvent.

### Advantages of the Present Invention

1. In the present process clethodim is prepared in the absence of a solvent.
2. The process is simple to handle and economically viable as there is a large amount of reduction in solvent volume.
3. The reaction time of the process is reduced which in turn reduces the operational cost.
4. The present continuous-flow process is simple, fast, high efficiency and easy operation.
5. The process is more environmentally friendly.
6. The present process provides final product in high yield and high purity.

The present invention has the advantage of short residence time of the material, high selectivity, high yield, less equipment investment, manufacturing cost savings, reduced material consumption, reducing the amount of by-products in the final product. Accordingly, the entire process is technically advanced over the conventional process, continuous, low energy consumption, an efficient and feasible continuous synthesis of clethodim. Thus, present process is simple, rapid and industrially viable.

Thus, the present invention involves technical advance as compared to the existing knowledge or having economic significance or both and that makes the invention not obvious to a person skilled in the art.

In accordance of the present invention clethodim is produced with a purity of greater than about 90%, 92%, 94%, 96% or greater than about 97%. In some embodiments, the purity is determined by high-performance liquid chromatography (HPLC) analysis. In some embodiments, clethodim produced according to the present invention is having purity more than 95% by HPLC.

The advantages and other parameters of the present invention is illustrated by the below given examples. However, the scope of the present invention is not limited by the examples in any manner. It will be appreciated by any person skilled in this art that the present invention includes aforesaid examples and further can be modified and altered within the technical scope of the present invention

### Examples

### Example 1

### Preparation of Clethodim in Batch Process

5-[2-(ethylthio)propyl]-2-(1-oxopropyl)-1,3-cyclohexanedione (161 g, 0.575 mole) was charged in a reaction flask. O-(3-chloro-2-propen-1-yl)hydroxylamine (70 g, 0.618 mole) was drop-wise added to it in 15 min at 25-30° C. and resulting reaction mixture was heated and stirred for 7 hr at 40-45° C. After completion of the reaction, the reaction mass was cooled to 20-25° C. 10% NaOH (250 ml) was added to the reaction mass and stirred for 30 min. To this mixture was added toluene (230 ml) and the layers were separated. The aqueous layer was acidified using 10% HCl (290 ml) and the product was extracted using toluene. The organic layers were combined, and the combined organic layer was washed with water, dried and concentrated under reduced pressure to obtain clethodim (203 g).

Yield: 94.88%

HPLC purity: 96.74%

### Example 2

### Preparation of Clethodim in a Batch Process

5-[2-(ethylthio)propyl]-2-(1-oxopropyl)-1,3-cyclohexanedione (37 g; 95.70%; 0.130 mole) was charged in a reaction flask. O-(3-chloro-2-propen-1-yl)hydroxylamine (18 g; 98.80%; 0.165 mole) was drop-wise added to it in 15 min at 25-30° C. and resulting reaction mixture was stirred for 2 h at 40-45° C. After completion of the reaction, the reaction was cooled to 20-25° C. To the mixture was added 10% NaOH (57 ml) and it was stirred for 30 min. To this mixture was added toluene (60 ml) and the layers were separated. The aqueous layer was acidified using 10% HCl (68 ml) and the product was extracted using toluene. The combined organic layer was washed with water, dried and concentrated under reduced pressure to obtain clethodim (46.0 g).

Yield: 94.50%

HPLC purity: 96.13%

### Example 3

### Comparative Example: Reaction in Dichloromethane

5-[2-(ethylthio)propyl]-2-(1-oxopropyl)-1,3-cyclohexanedione (13 g; 0.0464 mole) was charged in a reaction flask. O-(3-chloro-2-propen-1-yl)hydroxylamine (13 g; 0.0496 mole) in dichloromethane (7.6 g MDC+5.4 g CPHA) was drop-wise added in reaction flask in 15 min at room temperature and stirred it for 17 hr at 40-45° C. After completion of the reaction, the reaction is cooled to 20-25° C. To the mixture was added 10% NaOH (20 ml) was added to it to bring pH 12. Toluene (25 ml) was added to it and layers were separated. The aqueous layer was acidified using 10% HCl (23 ml) and the product was extracted using toluene (50 ml). The organic layer washed with water, dried and concentrated under reduced pressure to obtain clethodim (15 g).

Yield: 87.15%.

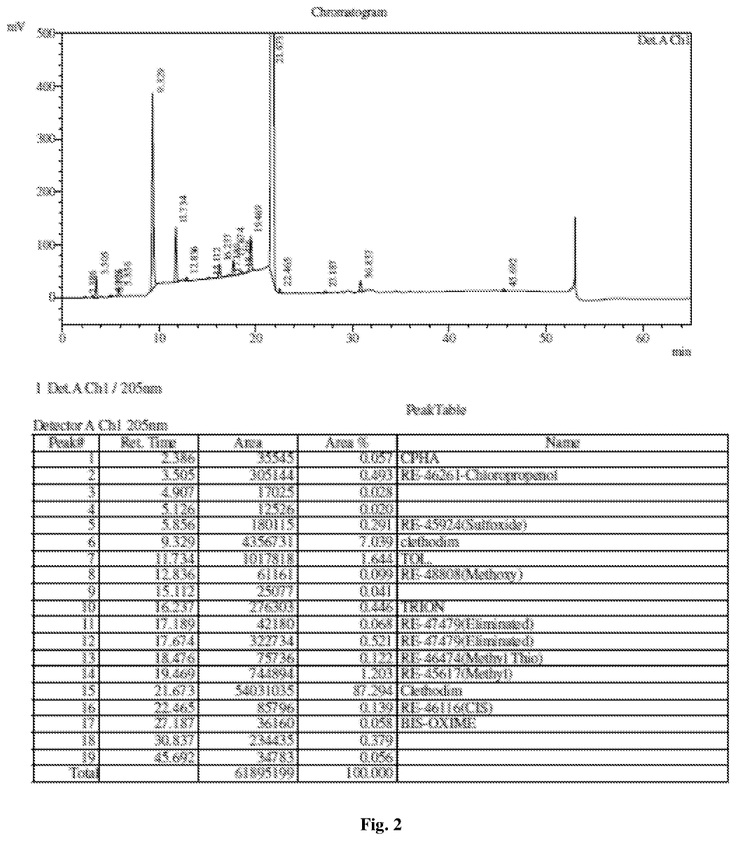

HPLC purity: 97.03% (FIG. 2).

### Example 4: Preparation of Clethodim in the Absence of Solvent in a Continuous Process

5-[2-(ethylthio)propyl]-2-(1-oxopropyl)-1,3-cyclohexanedione was fed to a two line of PFR (plug flow type reactor) by first dosing line (4) at the rate of 4.05 ml/min and O-(3-chloro-2-propen-1-yl)hydroxylamine was fed to the reactor by second dosing line (5) at the rate of 2.2 ml/min, the flow rate was adjusted to maintain stoichiometric ratio of 5-[2-(ethylthio)propyl]-2-(1-oxopropyl)-1,3-cyclohexanedione:O-(3-chloro-2-propen-1-yl)hydroxylamineat 1:1.37. The two lines discharge their contents in the reaction region (CP-01) which is maintained at 65° C. Clethodim was formed within residence time 8 min and collected in the collector vessel (3). The mass was treated with 10% sodium hydroxide (NaOH) and the aqueous layer was acidified with 10% hydrochloric acid (HCl). The product was then extracted using toluene. The combined toluene layer was distilled under vacuum to obtain clethodim. The process is represented in FIG. 1.

Yield: 95%.

HPLC Purity: 96.5%.

We Claim:

1. A process for the preparation of clethodim of formula (I) 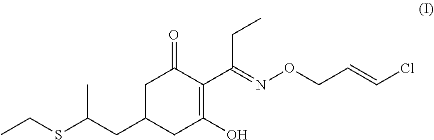 [SMILES: CCSC(C)CC1CC(=O)C(/C(CC)=N/OC/C=C/Cl)=C(O)C1]
by reacting 5-[2-(ethylthio)propyl]-2-(1-oxopropyl)-1,3-cyclohexanedione of formula (II) 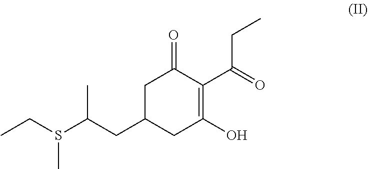
with O-(3-chloro-2-propen-1-yl)hydroxylamine of formula (III) 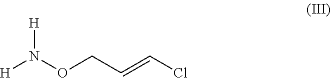
in the absence of a solvent.

2. The process according to claim 1 wherein 5-[2-(ethylthio)propyl]-2-(1-oxopropyl)-1,3-cyclohexanedione and O-(3-chloro-2-propen-1-yl)hydroxylamine are used in a molar ratio from about 1.0:1.0 to about 1.0:5.0.

3. The process according to claim 1 where the reaction is carried out at a temperature in the range from about 20° C. to about 70° C.

4. A continuous flow process for preparing clethodim, said process comprising:
a) charging 5-[2-(ethylthio)propyl]-2-(1-oxopropyl)-1,3-cyclohexanedione into a microreactor unit through a first dosing line;
b) charging O-(3-chloro-2-propen-1-yl) hydroxylamine into the microreactor unit through a second dosing line;
c) reacting 5-[2-(ethylthio)propyl]-2-(1-oxopropyl)-1,3-cyclohexanedione with O-(3-chloro-2-propen-1-yl) hydroxylamine in the absence of a solvent in a microreactor.

5. The process according to claim 4, wherein 5-[2-(ethylthio)propyl]-2-(1-oxopropyl)-1,3-cyclohexanedione flows from the first dosing line at a flow rate from about 1 ml/min to about 20 ml/min.

6. The process according to claim 4, wherein O-(3-chloro-2-propen-1-yl)hydroxylamine flows from the second dosing line at a flow rate from 1 ml/min to 20 ml/min.

7. The process according to claim 4, wherein the reaction is carried out at a temperature ranging from about 20° C.-to about 90° C.

8. The process according to claim 4, where the residence time for the reactants is from about 3 minutes to 15 minutes.

9. A system comprising a microreactor unit for producing clethodim by a continuous flow process as claimed in claim 4 wherein the process is carried out in the absence of a solvent.

10. Clethodim produced by a process comprising reacting 5-[2-(ethylthio)propyl]-2-(1-oxopropyl)-1,3-cyclohexanedione with O-(3-chloro-2-propen-1-yl)hydroxylamine (III) in the absence of a solvent.
